(19) United States
(12) Patent Application Publication
(10) Pub. No.: US 2006/0030869 A1
(43) Pub. Date: Feb. 9, 2006

(54) Adhesive anastomosis connection system

(75) Inventors: Amir Loshakove, Moshav-Bazra (IL); Ido Kilemnik, Herzelia (IL)

Correspondence Address: WOLF, BLOCK, SCHORR & SOLIS-COHEN LLP, 250 PARK AVENUE, NEW YORK, NY, 10177, US

(73) Assignee: BY-PASS, INC., Orangeburg, NY (US)

(21) Appl. No.: 10/535,383

(22) PCT Filed: Nov. 13, 2003
(86) PCT No.: PCT/IL03/00959

§ 371 Date: May 16, 2005

Related U.S. Application Data
(60) Provisional application No. 60/426,013, filed on Nov. 14, 2002.

(51) Int. Cl. A61B 17/08

(52) U.S. Cl. 606/153

(57) ABSTRACT

A method of performing an anastomosis, comprising, juxtaposing two blood vessels to be anastomosed using an juxtaposition device, to a desired configuration in which at least one vessel is an end vessel; applying an adhesive to said vessels while they are in said configuration, said adhesive being sufficient to ensure both sealing and bonding of said two vessels to each other, and removing said juxtaposition device after said adhesive sufficiently sets.

### RELATED APPLICATIONS The present application claims the benefit under 119 (e) of U.S. Ser. No. 60/426,013, filed on Nov. 14, 2002. The disclosure of which is incorporated herein by reference.

### BACKGROUND OF THE INVENTION

The problem of attaching two blood vessels is well known. While the gold standard is suturing, various connectors for attaching blood vessels have recently become more common.

U.S. Pat. No. 5,797,920 to Kim DuckSoo, describes a connector which is buttressed using an application of a tissue adhesive.

Tissue adhesives in general have found application in various types of surgical treatments, for example, surface cuts, GI tract procedures and small blood leaks near the heart.

U.S. Pat. No. 6,245,083 to Black, the disclosure of which is incorporated herein by reference, describes a device for apposing two side-by-side blood vessels for anastomosis. Bio-adhesive is then applied to the outside of the vessels. While the patent suggests that the method can be used for other types of connections, it is not clear how this suggestion can be carried out, as there is apparently no support in the disclosure and the procedures disclosed would not work for that suggestion. In addition, the adhesive is applied by a separate means from the apposition device.

U.S. Pat. No. 5,725,551 to Myers, et al teaches an adhesive based method for sealing punctures in blood vessels.

### SUMMARY OF THE INVENTION

A broad aspect of some embodiments of the invention relates to performing anastomotic connections using an adhesive. In an exemplary embodiment of the invention, the resulting connection is either a side to end or end to end connection. In some embodiments of the invention, a scaffold is used during the procedure, for example to ensure that the vessels are properly located with respect to each other, but may be removed and/or dissolve later and/or serve little or no structural purpose once the adhesive has set. Optionally, a connector is provided, to work in conjunction with the adhesive. Optionally, the adhesive is used to protect against possible shortcomings of the connector. For example, the connector may provide initial strength and the adhesive long term strength and/or sealing. Optionally, a combined scaffolding and adhesive delivery system is used to hold the vessels and apply the adhesive.

In exemplary embodiments of the invention, an adhesive provides one or both of the following functions, bonding the two vessels and sealing the attachment area between the vessels. In an exemplary embodiment of the invention, the adhesive both bonds and seals.

In an exemplary embodiment of the invention, an anastomosis scaffolding comprises a scaffolding, such as one or more wires which are pulled through the adhesive, before, during or after the setting of the adhesive. This scaffolding may be, for example outside the blood vessel, optionally piercing one or both blood vessels.

Alternatively or additionally, an anastomosis scaffolding comprises a support inside a blood vessel, for example one or more inflatable or expandable structures such as balloons.

In an exemplary embodiment of the invention, an adhesive delivery system remains outside of the lumens of the blood vessels being attached. Optionally, any scaffolding also remains outside of these lumens.

In an exemplary embodiment of the invention, the device scaffolding is designed to support a range of angles between the two blood vessels (e.g., including grafts) that are being attached to each other.

An aspect of some embodiments of the invention relates to an anastomosis connection system, adapted to connect a side vessel to an end vessel using adhesive. In an exemplary embodiment of the invention, the system includes an internal scaffold, for example a balloon and/or a deployable element. Optionally, the internal scaffold is inserted through an incision one of the blood vessels, which may later be sealed, if required.

An aspect of some embodiments of the invention relates to an end to end anastomosis system, using adhesive, in which any scaffold is external to the blood vessels being attached to each other. In an exemplary embodiment of the invention, the system comprises two vessel holders, each of which holds one vessel, optionally with everted lips. The two holders are then brought together and the connection made, for example by application of adhesive.

An aspect of some embodiments of the invention relates to an anastomosis system including pullers for pulling and holding two blood vessels to be connected and an adhesive applicator to apply an adhesive to the anastomosis area.

A broad aspect of some embodiments of the invention relates to techniques for adhesive based anastomosis. In an exemplary embodiment of the invention, an anastomosis adhesive system includes one or more blowers, which may be used, for example, to dry an area in preparation for adhesive application and/or for assistance in setting the adhesive.

In an exemplary embodiment of the invention, the adhesive system includes one or more nozzles, for example a ring array of nozzles. Optionally, the nozzles are used for controlled application of adhesive. In an exemplary embodiment of the invention, the nozzles are arranged to preferentially provide more adhesive at one side than another, for example if an oblique anastomosis is performed and/or depending at the instant angle at the area of application between the two vessels.

Optionally, adhesive shaping is provided using a form that controls the extent of adhesive flow. Optionally, a flow limiter is provided, which prevents overflow of adhesive to adjacent tissue. Such a flow limiter may include, for example, a collar and/or a tissue protector pad.

In an exemplary embodiment of the invention, means for removing bubbles from an adhesive globule is provided, for example, one or more thin needle nozzles attached to a suction source. Optionally, the needles are provided in the path of adhesive between a delivery nozzle to an anastomosis area and/or inside the anastomosis area.

Optionally, a foaming means is provided, for example a gas source to be mixed in with the adhesive or a specially shaped nozzle. Such foam may adhere better and/or dry better.

In an exemplary embodiment of the invention, a primer layer is applied prior to applying adhesive, for example on an adventisia part and/or on an intima part of blood vessels. Different primers may be provided for different tissue types. Optionally, a hardener is provided after the adhesive. Optionally, different nozzles and/or sets of nozzles are used for different applied materials. If a binary adhesive is used, the binary components may share a single nozzle and/or supply tube or be mixed after application. Optionally, one or more of the adhesive compounds, adhesives area and/or other delivered materials are heated by the anastomosis system, for example, to hasten setting.

In an exemplary embodiment of the invention, the delivery system utilizes electrical and/or mechanical mechanism for timing the operation of the system, for example application of adhesive, blow drying and release of the completed connection after the adhesive is sensed and/or estimated to be dry. Optionally, a visual marker is used, for example a slowly oxidizing dye, which changes color after a set time period corresponding to the setting time of the adhesive, so that an operator can determine if a scaffolding may be removed.

A particular feature of some embodiments of the invention which may make them easier to use is that blood flow can continue during the adhesive setting time and/or the vessel attachment time.

An aspect of some embodiments of the invention relates to holding an end of a vessel open, from the outside, using vacuum, for example using one or more vacuum nozzles arranged adjacent its circumference at one end. Optionally, the nozzles, when activated, maintain the vessel in an everted or semi-everted position.

An aspect of some embodiments of the invention relates to a method of attaching two vessels in which one or more scaffold elements, for example one or more wires, are adhered, for example using an tacky glue to the outside of one or both vessels. Then the vessels are attached, for example using adhesive, and the wires pulled out. Alternatively or additionally, the wires are hooked into the vessel(s).

In an exemplary embodiment of the invention, the wires are coated with a material that at least partially resists adhesion by the adhesive. Optionally, the wires are heated or vibrated to assist their removal.

An aspect of some embodiments of the invention relates to using an internal shunt as a scaffolding for an adhesive anastomotic procedure. Optionally, the shunt is somewhat stiffened, to ensure that it holds the vessels. Unlike standard shunts for anastomosis, in which a region is provided near the anastomosis area for needle maneuvers, in an exemplary embodiment of the invention, the shunt fills the blood vessels at the anastomotic area, to provide support. As the anastomosis is performed by applying an adhesive outside the blood vessels, in some embodiments of the invention the shunt does not interfere with needle manipulation of vessel manipulation, as they are not needed.

There is thus provided in accordance with an exemplary embodiment of the invention, a method of performing an anastomosis, comprising:

juxtaposing two blood vessels to be anastomosed using an juxtaposition device, to a desired configuration in which at least one vessel is an end vessel;

applying an adhesive to said vessels while they are in said configuration, said adhesive being sufficient to ensure both sealing and bonding of said two vessels to each other; and

removing said juxtaposition device after said adhesive sufficiently sets. Optionally, juxtaposing comprises engaging at least one of said vessels using said juxtaposition device

In an exemplary embodiment of the invention, juxtaposing comprises inserting at least a portion of said juxtaposing device into at least one of said vessels.

In an exemplary embodiment of the invention, juxtaposing comprises inserting at least a portion of said juxtaposing device into a wall of at least one of said vessels.

In an exemplary embodiment of the invention, juxtaposing comprises juxtaposing by manipulating said juxtaposing device. Optionally, juxtaposing comprises pulling at least one of said vessels using said juxtaposing device.

In an exemplary embodiment of the invention, juxtaposing comprises juxtaposing said vessels to have substantially no space between the vessels at an anastomotic location thereof. Optionally, juxtaposing comprises juxtaposing said vessels to have a sealed contact therebetween.

In an exemplary embodiment of the invention, juxtaposing comprises providing at least one of said vessels using a graft delivery system and wherein said juxtaposition device is coupled to said graft delivery system.

In an exemplary embodiment of the invention, applying an adhesive comprises applying an adhesive to said juxtaposition device.

In an exemplary embodiment of the invention, applying an adhesive comprises not applying an adhesive to said juxtaposition device.

In an exemplary embodiment of the invention, the method comprises drying an anastomosis area of said blood vessels using a stream of air, prior to said applying.

In an exemplary embodiment of the invention, the method comprises assisting a setting of said adhesive. Optionally, said assisting comprises blowing air on said adhesive. Optionally, said air is heated.

In an exemplary embodiment of the invention, applying comprises mixing an adhesive from at least two components during a process of flowing said adhesive during said applying.

In an exemplary embodiment of the invention, applying comprises applying using at least one nozzle.

In an exemplary embodiment of the invention, applying comprises applying using a plurality of nozzles arranged in a ring.

In an exemplary embodiment of the invention, applying comprises applying into a mold adjacent said anastomosis area, which mold shapes the adhesive about said area

In an exemplary embodiment of the invention, applying comprises into a form adapted to inhibit spillover.

In an exemplary embodiment of the invention, applying comprises applying a pre-measured amount of adhesive.

In an exemplary embodiment of the invention, applying comprises applying as a continuous flow.

In an exemplary embodiment of the invention, the method comprises removing some of said adhesive after said applying.

In an exemplary embodiment of the invention, said removing comprises removing after said adhesive sets completely.

In an exemplary embodiment of the invention, said removing comprises removing before said adhesive sets completely.

In an exemplary embodiment of the invention, said removing comprises removing as soon as said adhesive starts to set.

In an exemplary embodiment of the invention, said removing comprises removing before said adhesive starts to significantly set.

In an exemplary embodiment of the invention, said applying comprises applying while blood flows in at least one of said blood vessels.

In an exemplary embodiment of the invention, no foreign materials other than said adhesive remain in said anastomosis.

In an exemplary embodiment of the invention, the method comprises providing at least one strengthening element in said anastomosis and leaving said at least one strengthening element permanently in said anastomosis.

There is also provided in accordance with an exemplary embodiment of the invention, a method of performing an anastomosis, comprising:

attaching at least a first scaffold element to a first blood vessel;

attaching at least said first scaffold element or a second scaffold element to a second blood vessel;

positioning said blood vessels using said at least a first scaffold element, to a desired configuration;

applying an adhesive to said vessels while they are in said configuration, said adhesive being sufficient to ensure both sealing and bonding of said two vessels to each other; and

removing said at least first scaffolding element.

In an exemplary embodiment of the invention, said first blood vessel is a side vessel. Alternatively, said first blood vessel is an end vessel.

In an exemplary embodiment of the invention, the method comprises mechanically attaching said at least first scaffolding element to said first blood vessel. Optionally, mechanically attaching comprises piercing. Alternatively or additionally, mechanically attaching comprises hooking.

In an exemplary embodiment of the invention, the method comprises adhesively attaching said at least a first scaffolding element to said first blood vessel. Optionally, the method comprises adhesively attaching using a tacky adhesive.

In an exemplary embodiment of the invention, the method comprises attaching a second scaffolding element to said second blood vessel.

In an exemplary embodiment of the invention, a same first scaffolding element is attached to both of said blood vessels.

In an exemplary embodiment of the invention, said at least one scaffolding element comprises a plurality of wires.

In an exemplary embodiment of the invention, said at least one scaffolding element comprises a shunt.

In an exemplary embodiment of the invention, said at least one scaffolding element comprises a balloon.

In an exemplary embodiment of the invention, positioning comprises direct manual positioning.

In an exemplary embodiment of the invention, positioning comprises positioning by manipulating said at least one scaffolding.

In an exemplary embodiment of the invention, positioning comprises positioning said vessels to have substantially no space between the vessels at an anastomotic location thereof.

In an exemplary embodiment of the invention, applying an adhesive comprises applying an adhesive using a port coupled to said at least one scaffolding element.

In an exemplary embodiment of the invention, applying an adhesive comprises applying an adhesive into a form which shapes said adhesive about said configuration.

In an exemplary embodiment of the invention, the method comprises blowing air on said adhesive to aid setting.

In an exemplary embodiment of the invention, removing comprises removing said at least one scaffolding element after said adhesive sets.

In an exemplary embodiment of the invention, removing comprises removing at least one of said at least one scaffolding elements before said adhesive sets.

There is also provide din accordance with an exemplary embodiment of the invention, apparatus comprising:

an adhesive source;

at least one adhesive delivery port;

at least one blood vessel holder adapted to stabilize said port relative to an anastomosis area of two blood vessels.

In an exemplary embodiment of the invention, said vessel holder is adapted to remain external to said vessels.

In an exemplary embodiment of the invention, said vessel holder is adapted to penetrate at least one of said vessels.

In an exemplary embodiment of the invention, said vessel holder is adapted to penetrate both of said vessels.

In an exemplary embodiment of the invention, said vessel holder comprises a plurality of wires adapted to engage a blood vessel.

In an exemplary embodiment of the invention, said vessel holder comprises a vacuum source which applies vacuum to an outside of a vessel, thereby holding it.

In an exemplary embodiment of the invention, the apparatus comprises a rim adapted to inhibit spillover of adhesive.

In an exemplary embodiment of the invention, said apparatus defines a form between said apparatus and said blood vessels, for filling with adhesive and shaping a desired set adhesive configuration.

In an exemplary embodiment of the invention, said at least one delivery port comprises a plurality of ports adapted to be arranged around one of said vessels

In an exemplary embodiment of the invention, said at least one delivery port comprises a structure adapted to mix at least two components of an adhesive.

In an exemplary embodiment of the invention, said at least one delivery port comprises at least one nozzle attached to a first component source of adhesive and at least one nozzle attached to a second component source of adhesive.

In an exemplary embodiment of the invention, the apparatus comprises a source of gas adapted to be aimed at said anastomosis area.

In an exemplary embodiment of the invention, the apparatus comprises means for assisting setting of said adhesive.

In an exemplary embodiment of the invention, the apparatus comprises a heater for said gas.

In an exemplary embodiment of the invention, the apparatus comprises a setting sensor which provides an indication of setting of said adhesive.

In an exemplary embodiment of the invention, the apparatus comprises a setting timer.

In an exemplary embodiment of the invention, the apparatus comprises a controller which synchronizes a an activation of said vessel holder and delivery of said adhesive. Optionally, said controller is a mechanical controller using mechanical means to determine said synchronization. Alternatively, said controller is an electrical controller using circuitry to determine said synchronization.

In an exemplary embodiment of the invention, said apparatus is mounted in a delivery capsule adapted to be attached to a delivery system and adapted to hold a graft vessel therein.

In an exemplary embodiment of the invention, said capsule includes a set amount of adhesive. Alternatively or additionally, said capsule includes a single external control.

In an exemplary embodiment of the invention, said vessel holder is arranged to be split after use.

In an exemplary embodiment of the invention, said vessel holder includes a slot in a side thereof, adapted for placement of a vessel therein. Optionally, said slot is fixed.

There is also provided in accordance with an exemplary embodiment of the invention, an adhesive anastomotic system, comprising:

a first blood vessel holder;

a second blood vessel holder adapted to interlock with said first blood vessel holder, such that blood vessels held by said two vessel holders contact; and

an adhesive port configured to deliver an adhesive to said contact.

Optionally, said vessel holders are configured to hold two vessels in an end-to-end configuration.

There is also provided in accordance with an exemplary embodiment of the invention, apparatus comprising:

an adhesive source;

at least one adhesive delivery port;

at least one vessel holder mechanically coupled to said delivery port and adapted to stabilize said port relative to at least one blood vessel; and

an adhesive setting enhancer. Optionally, said enhancer comprises a source of gas. Alternatively or additionally, said enhancer comprises a source of energy.

### BRIEF DESCRIPTION OF THE DRAWINGS

Exemplary, non-limiting embodiments of the invention will be described below, with reference to the following figures, in which the same elements are marked with the same reference numbers in different figures:

FIG. 1 is a flowchart of a method of performing an adhesive-based anastomosis, in accordance with an exemplary embodiment of the invention;

FIGS. 2A-2G are a series of illustrations showing various stages in the application of the method of FIG. 1 to a side to end anastomosis;

FIG. 3 is a schematic illustration of a vacuum based graft delivery system, in accordance with an exemplary embodiment of the invention;

FIGS. 4A-4E illustrate hook-based scaffolding, in accordance with an exemplary embodiment of the invention;

FIGS. 5A-5C illustrate adhesive-based scaffolding, in accordance with an exemplary embodiment of the invention;

FIG. 6 is a schematic illustration of an adhesive delivery system, in accordance with an exemplary embodiment of the invention;

FIG. 7 shows an adhesive delivery system including a rim, in accordance with an exemplary embodiment of the invention.

FIGS. 8A-8E show various attachment configurations of blood vessels, in accordance with exemplary embodiments of the invention;

FIGS. 9A and 9B show a method of achieving the configuration of FIG. 8C, in accordance with an exemplary embodiment of the invention;

FIGS. 10A and 10B show a method of achieving the configuration of FIG. 8E, in accordance with an exemplary embodiment of the invention;

FIGS. 11A-11G show nozzle designs, in accordance with exemplary embodiments of the invention;

FIGS. 12A-12C illustrate an end-to-end anastomosis, in accordance with an exemplary embodiment of the invention;

FIGS. 13A-13D are side cross-sectional views showing embodiments where a support is provided inside the blood vessels, in accordance with exemplary embodiments of the invention; and

FIG. 14 is a cross-sectional view of an adhesive anastomosis capsule, in accordance with an exemplary embodiment of the invention.

### DETAILED DESCRIPTION OF EXEMPLARY EMBODIMENTS

Overview of Adhesive Anastomosis

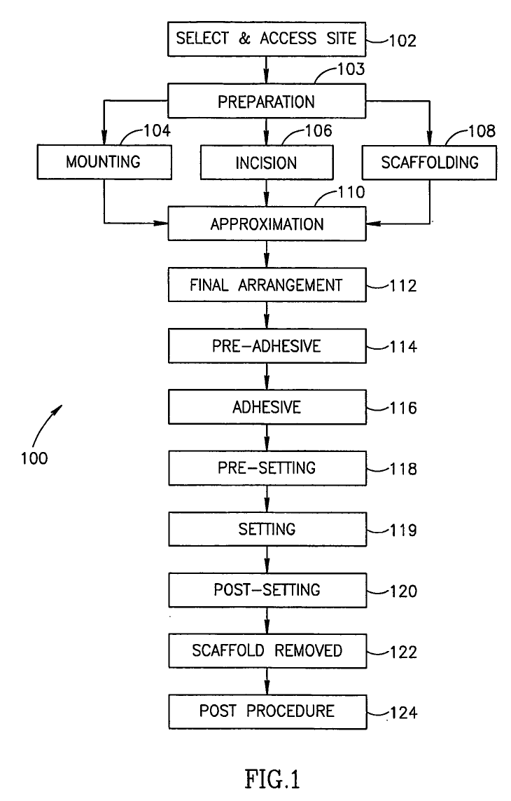

FIG. 1 is a flowchart of a method of performing an adhesive-based anastomosis, in accordance with an exemplary embodiment of the invention. This flowchart is described below in conjunction with various exemplary variations of adhesive based anastomosis.

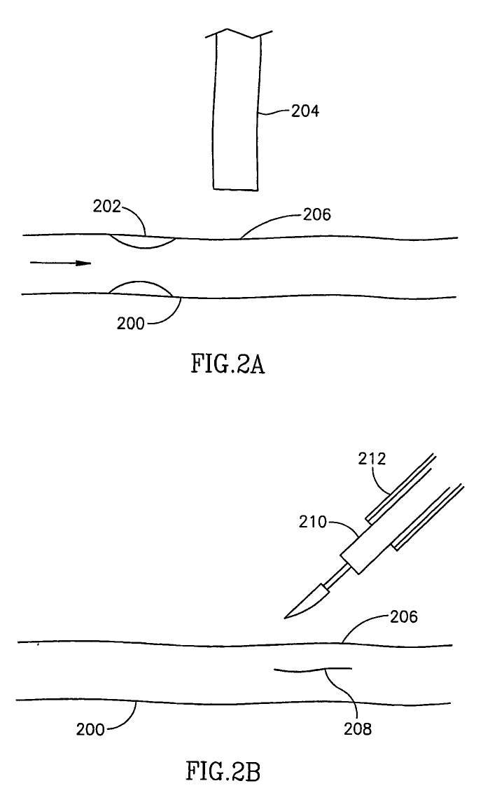

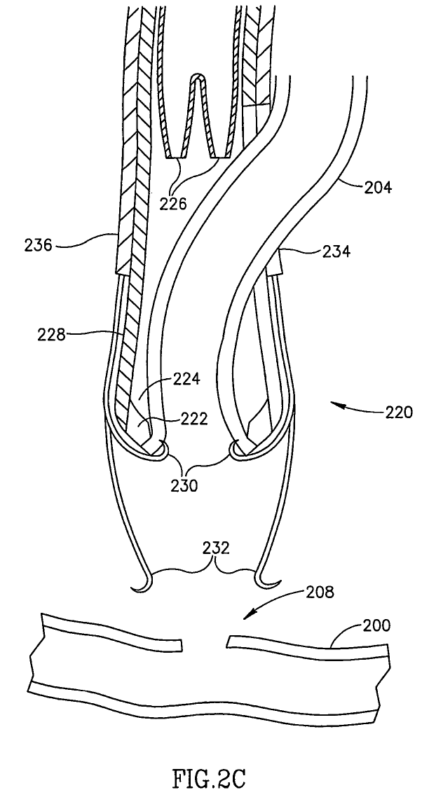

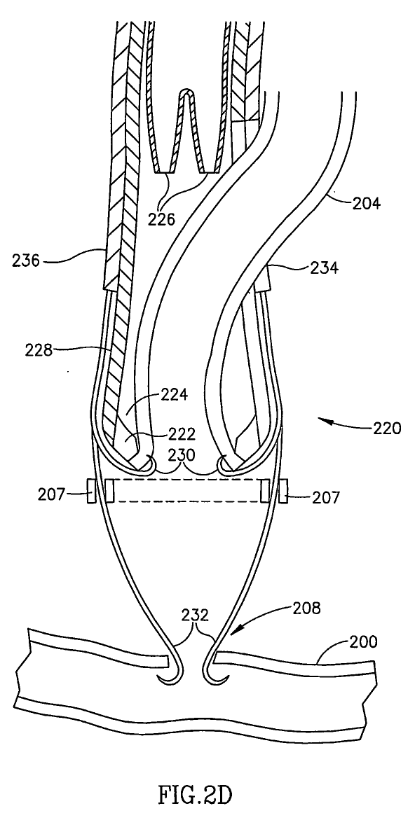

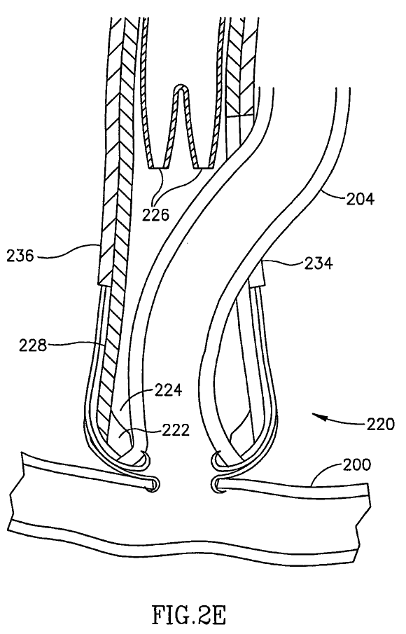

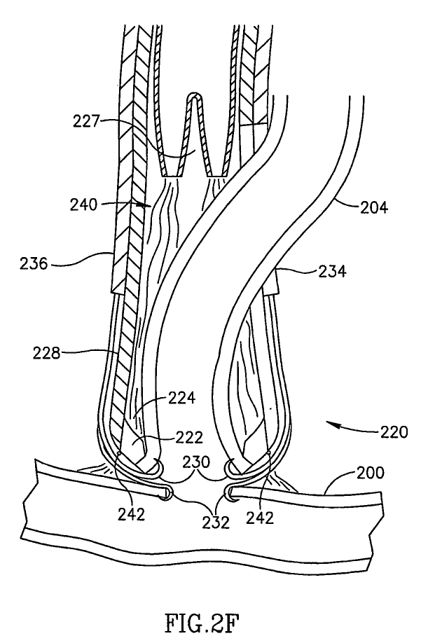

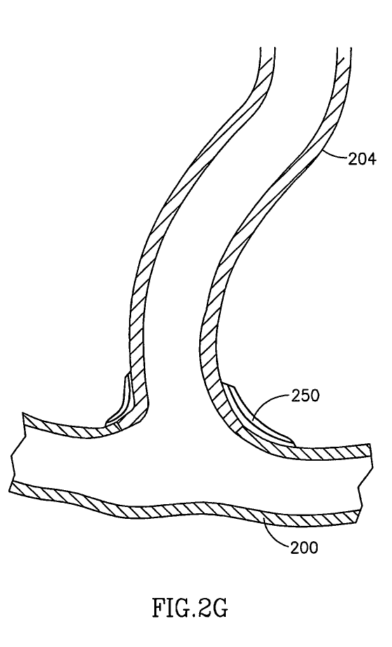

FIG. 2A shows a “side” vessel 200, for example a coronary vessel, including an occlusion 202, for example partial or complete. In a bypass procedure, a graft 204, for example a patent blood vessel, such as the ITA (LIMA, RIMA), Radial, GEA, or a live graft, such as saphenous vein, a xenograft or an artificial graft is attached to the side of vessel 200 at a point 206. In the example of a vessel other than the LIMA and RIMA, the other end of the vessel may be connected to a source of blood, such as the aorta or the other side of occlusion 202. While not essential, in an exemplary embodiment of the invention, the attachment is a side (of vessel 200) to end (of graft 204) attachment. Unless otherwise specified, the terms blood vessel and graft are used interchangeably in this specification and its claims. However, it should be noted that the adhesion properties and resistance to transfixing and cutting is different for different types of blood vessels and grafts and may affect various device parameters.

Procedure Preamble

There are many clinical indications for the need to do an anastomosis, for example damaged blood vessel, clogged or stenosed blood vessels and organ or tissue implantation. For clarity, the example used herein is that of anastomosis as part of a coronary bypass procedure, specifically, anastomosis of a LIMA or RIME to a coronary artery, at a point below occlusion 202. However, the scope of the invention is not to be limited solely to methods and apparatus for these particular procedures and is applicable to other anastomosis procedures.

Depending on the particular procedure and the situation of the patent, the location at which the anastomosis is to be made, is selected (102). In addition, various approaches to the site may be used. For example, a heart can be reached by opening the chest and spreading the ribs. Alternatively or additionally, the heart can be reached using a keyhole approach with flexible or rigid tool. Alternatively or additionally, a transvascular approach may be used. Various apparatus suitable for these different approaches are described below.

Preparation (103)

A blood vessel is typically covered with various tissue layers, some of which may not be compatible (e.g., adhere well) with the adhesive used. Alternatively or additionally, some of the layers may be incompatible with a patent connection, for example, not being sealed to blood or naturally flaking. For example, an adventisia layer may not attach well to an intima layer. Alternatively or additionally, one of the vessels is a non-living graft, for example, being made of polyester.

In an exemplary embodiment of the invention, the surface of one or both of the target vessels (e.g., near where adhesive is expected to be applied) is prepared, for example by cleaning, drying or applying a primer. Exemplary primers include a setting enhancer, a coagulation enhancer, a fibrin layer and/or bio-active materials which affect the healing and/or surface characteristics (e.g., pore opening) of the target tissue. Alternatively or additionally, such preparation is provided later, for example as described below. Alternatively or additionally, the anastomosis area may be washed, for example using a saline solution, optionally provided from one or more of the adhesive nozzles (described below).

Blood flow to one or both of the vessels may be stopped (if it exists) prior to incision, for example, using clamps. Alternatively, flow in at least the side vessel is allowed during the anastomotic procedure, for example, by covering the incision with a finger and then quickly inserting vessel 204 into the incision, possibly covering the incision with a finger in the mean-time.

Incision (106)

Typically, an aperture needs to be formed in one or both of the target vessels (104). In an exemplary embodiment of the invention, an incision is formed. Exemplary systems for forming openings (e.g., by cutting or punching) in blood vessels are described, for example in PCT publications WO 01/70090, WO 02/30172, WO 99/62415, WO 01/0091, WO 01/41623, WO 00/56228, WO 02/074188, WO 02/47532, WO 02/47561, WO 00/56226, to applicant ByPass inc., the disclosures of which are incorporated herein by reference. Other arteriotomes are known as well in the art and may be used. In some embodiments of the invention, other means, such as laser cutters are used to form openings. The incision forming device may be separate from the adhesion device. Alternatively, they may be integrated. For example, a single delivery tube may be used selectively for providing a hole punch or vessel cutter and then providing a graft mounting system. Optionally, incisions are used for smaller vessels and punching is used for larger vessels.

FIG. 2B shows vessel 200 (in side view) with an incision 208 formed therein, and an exemplary cutter 210 suitable for forming such an incision. Cutter 210 is optionally provided through a delivery system 212, for example a flexible or rigid tube. Alternatively to forming an incision, a hole may be punched. In an exemplary embodiment of the invention, the punch of PCT publication WO 99/62415 is used, which punch forms a seal in the blood vessel. Also, this punch allows the punching element to be removed and a graft delivery system provided, without significant leakage of blood.

While an end vessel does not generally need an incision, some preparation may be provided, for example ensuring the angle of the vessel is suitable or cutting off ragged parts.

Mounting (104)

FIG. 2C shows vessel 204 mounted (104) in a graft delivery system 220, which may itself be provided via tube 212 (shown in 2B), in accordance with one exemplary embodiment of the invention. In the particular example shown, vessel 204 is partially everted or flared over a lip 222. In an exemplary embodiment of the invention, lip 222 comprises a plurality of lip elements that jut radially inwards from a tip of a delivery tube 228, thus allowing adhesive to flow between the elements (e.g., lips 222 may be formed as a slotted ring). Also shown are one or more nozzles 226 for providing an adhesive which flows through a volume 224 defined between the graft and delivery tube 228. In an exemplary embodiment of the invention, tube 228 prevents adhesive from spilling out of the anastomosis area, for example as will be described below.

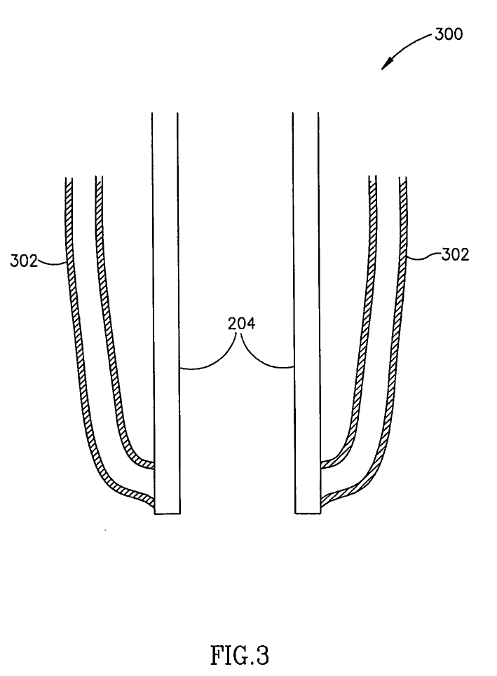

Also shown are a plurality of pullers 230 which serve to hold vessel 204 in place and/or to assist in flaring of vessel 204, it should be noted that in some embodiments of the invention, vessel 204 is not everted. Alternatively or additionally, if a temporary connector is provided, the connector itself may hold the graft, for example as described in the above applications. Alternatively or additionally, lip elements 222 may include barbs (not shown) to hold vessel 204. FIG. 3 shows a graft delivery system 300 in accordance with an alternative embodiment of the invention, in which a plurality of suction elements 302 together hold the tip of vessel 204. This type of suction system may be used for anastomosis methods that do not use adhesives, as well. Suction is typically provided by suction sources or by a wall socket in operating rooms.

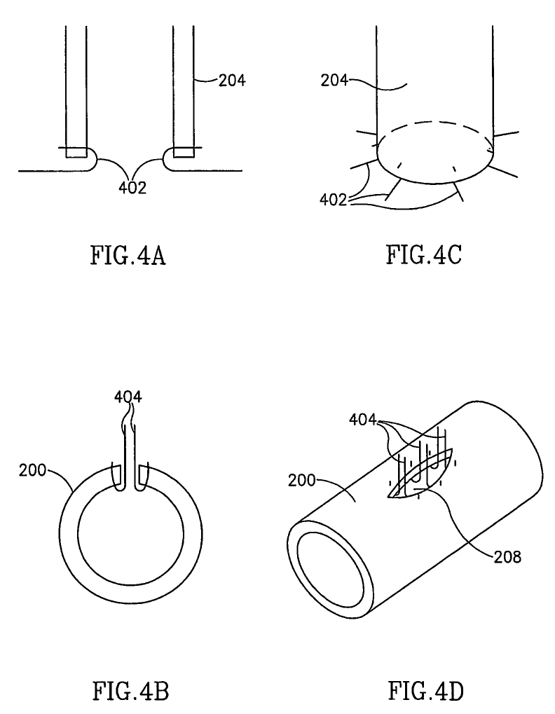

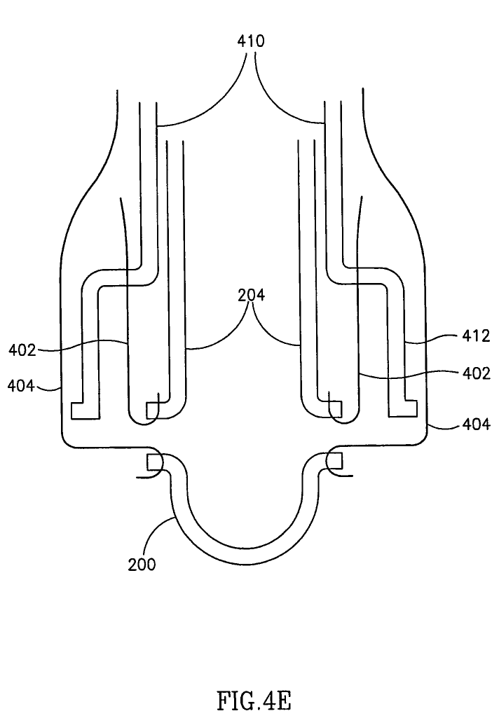

Returning to FIG. 2C, also shown are a plurality of pullers 232 which are optionally used for guiding vessel 204 to vessel 200 and/or for widening incision 208. PCT publications WO 01/41624, WO 01/70090 and WO 02/30172, for example, the disclosures of which are incorporated herein by reference, describe anastomosis system delivery systems including one, two or more sets of pullers. In some embodiments of the invention, an anastomosis connector is provided to hold together the blood vessels and the adhesive serves as a sealant and possibly for strengthening. Alternatively or additionally, the connector is used to hold the vessels together and then removed. One suitable removable connector is a clip ring shown in FIG. 4 of PCT publication WO 01/70090, which may be coupled to the delivery system, for example by welding. This clip ring comprises a ring having fingers extending away from the ring in a manner which allows the delivery system to close the fingers from outside the blood vessel in the shape of clips. The ring is optionally split, to assist removal.

In an exemplary embodiment of the invention, vessel 204 is mounted by pulling vessel 204 through an aperture 234 in the side of graft delivery system 220. An exemplary pulling system is shown in PCT publication WO 01/70118, the disclosure of which is incorporated herein by reference. Then, pullers 230 are loosened and placed on the tip of vessel 204, for example manually or using tweezers. Pullers 230 are then tightened, for example by retraction of a puller retractor 236, completing the mounting.

Approximation (110)

Vessel 204 is guided to a correct alignment with vessel 200, the term juxtapose (and juxtaposition) are also used herein as the process (and state) where two blood vessels are positioned one near another in a configuration. Guiding may be by manipulating pullers 232. Alternatively or additionally, guiding is directly manual, for example holding and moving one or both vessels by hand or using a tool which is a direct extension of the hand, such as forceps or manipulating a guide tube that holds a vessel, thereby being a direct extension of the vessel. FIG. 2D shows pullers 232 extended and inserted into incision 208. In an exemplary embodiment of the invention, approximation includes opening incision 208 so that some of delivery system 220 and/or vessel 204 may enter. Opening the incision may be achieved automatically by retracting pullers 232. For example, an optional apertured ring 207 may be provided, which urges pullers 232 apart. Alternatively or additionally, one or more external arms (not shown) may be used to selectively radially separate pullers 232.

PCT application PCT/IL02/00790, the disclosure of which is incorporated herein by reference describes a hook guide which may be used to guide the tips of pullers 232 into incision 208. In brief, the guide comprises, in one embodiment thereof, a tube slotted along its length with a flared end and a pointed end and having an inner lumen sufficient to receive the tips of pullers 232. The pointed end is placed into incision 208 and then the tips of pullers 232 are guided into the tube, along the slit, using the flaring for assistance. The bodies of pullers 232 remain outside the tube and pass through the slot into the tube lumen. When the puller tips are inside incision 208, the tube is removed.

Final Arrangement (112)

When pullers 232 are retracted as compared with the side vessel, the two vessels are arranged and, optionally, are not allowed to move relative to each other until after the adhesive is set. FIG. 2E shows vessels 200 and 204 in the final arrangement configuration and prior to application of adhesive. In addition, in the embodiment shown, pullers 232 also widen incision 208, as they extend radially. As shown, the pullers are not retracted enough to contact the intima of vessel 200 to the intima of vessel 204, however, in other embodiments this may be done. Similarly, while vessel 204 is partially-everted/flared to expose its intima, in some embodiments, it is not

In some embodiments of the invention, a temporary anastomotic connector or clip(s) is used to perform this final arrangement. The connector may then be removed or dissolve on its own, for example. In other embodiments, the stabilization of the blood vessels is provided by maintaining the pullers in a fixed configuration. In some cases, if the pullers are moved, the vessels move away from arrangement.

Scaffolding (108)

Alternatively or additionally to using a suitable delivery system, other methods may be used to bring vessels 200 and 204 to the situation of FIG. 2E. In an exemplary embodiment of the invention, scaffolding is mounted on one or more of vessels 200 and 204 and then used for approximation and/or attachment.

FIGS. 4A-4E illustrate hook-based scaffolding, in accordance with an exemplary embodiment of the invention. FIGS. 4A and 4B side cross-sectional views showing the mounting of a plurality of scaffold wires 402 and 404 on vessels 204 and 200, respectively. FIGS. 4C and 4D are perspective views of vessels 204 and 200, respectively. FIG. 4E is a side-cross-sectional view showing vessels 200 and 204 after approximation and attachment. After the anastomosis is completed, the scaffolding is removed, for example, by pulling, as described below.

The scaffolds may be manipulated, for example, by hand or using simple tools such as pliers, for mounting and movement of the blood vessels. Alternatively, the scaffolds may be mounted onto a guidance system, for example a system 410 shown in FIG. 4E. The mounting may be performed, for example after the scaffolds hook onto the vessels. System 410 comprises generally of a tube, having a flaring tip 412 with a rim at one end thereof, which rim defines one or more slots or apertures to hold the scaffold wires, when the wires are bent back towards the other end of system 410. An engagement ring (not shown) may be used to selectively pull back some or all of the scaffold wires and/or to hold them in place. Such a ring may be, for example, an elastic band on the tube.

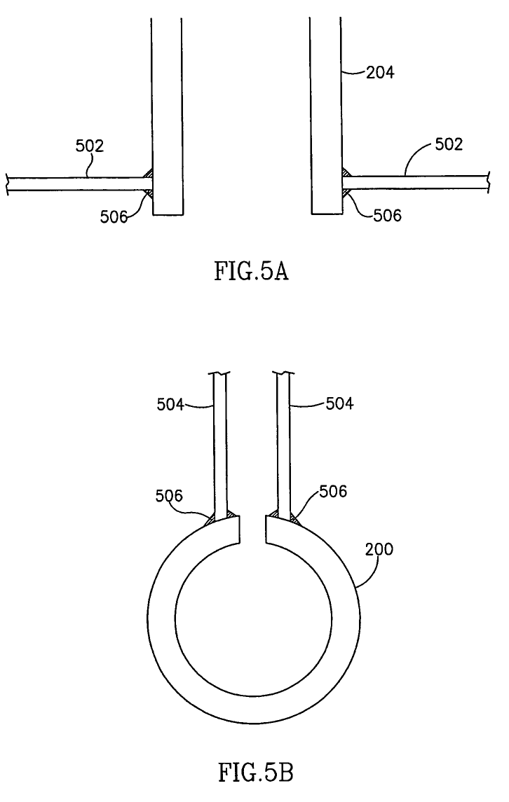

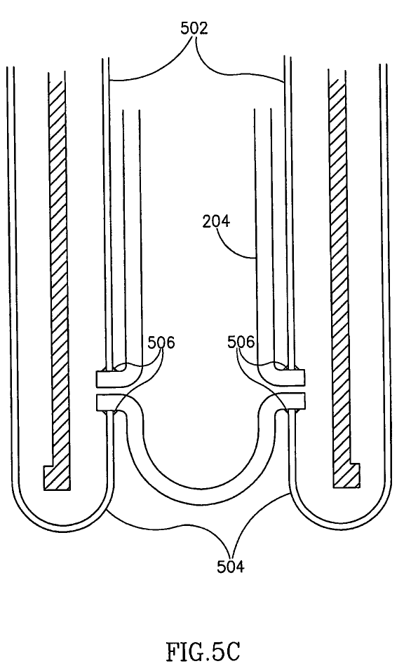

FIGS. 5A-5C illustrate adhesive-based scaffolding, in accordance with an exemplary embodiment of the invention. FIGS. 5A and 5B are cross-sectional views of scaffold wires 502 and 504 mounted using adhesive dabs 506 to vessels 204 and 200 respectively. This scaffolding may be mounted, for example by hand, optionally by applying one or more suture. Alternatively or additionally, a scaffold delivery system is provided, for example, a tube coated with an adhesive layer (not shown), with the scaffolding mounted on its end, possibly a thick tacky layer which optionally does not dry. After the scaffolding is contacted to vessel 200, the tube is removed, leaving the scaffolding. The tube is optionally protected against inadvertent contact with nearby tissues, by an outer tube.

It should be noted that in different embodiments, the acts of mounting (104), incision (106) and scaffolding (108) may be applied in other orders.

In an exemplary embodiment of the invention, the scaffolding of vessel 200 is then attached to delivery system 220. Optionally, this allows the scaffolding to be used, by the delivery system, for guidance and/or attaching the vessels.

While the scaffolds are shown as uncoupled wires, this is not essential. For example, the scaffold may comprise an apertured ring (not shown) through which apertures the wires pass and which contacts one or both blood vessels when the vessels are attached.

In an alternative exemplary embodiment of the invention, the scaffolding comprises one or more sutures, which may then dissolve or be pulled out, after their function is completed, for example.

Pre-Adhesion (114)

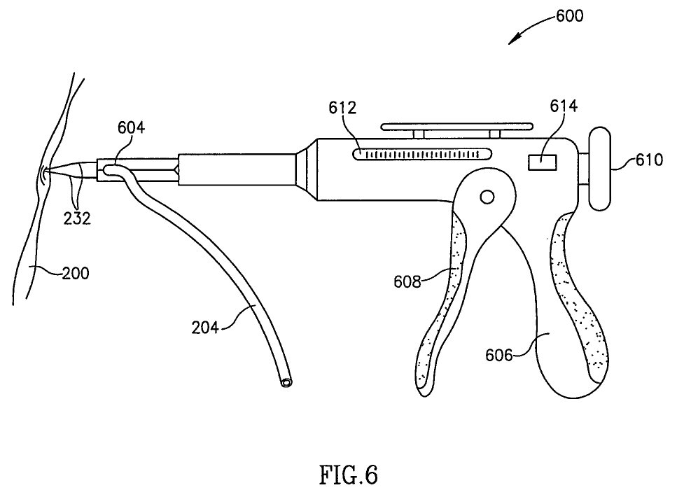

In an exemplary embodiment of the invention, before an adhesive is applied, the anastomotic area may be prepared (114), for example, by providing a primer as described above. One way of applying a primer is using the adhesive applicator described below in FIG. 6. Alternatively or additionally, the anastomotic area may be dried, for example by applying a stream of gas.

Adhesion (116)

FIG. 2F shows an adhesive material 240 being applied to the anastomotic area (116), in accordance with an exemplary embodiment of the invention. While the term adhesive is used, various embodiments of the invention utilize different bonding and sealing effects. For example, the adhesive may be provided outside the blood vessels to seal in any blood and/or to maintain the two vessels in correct approximation. Alternatively or additionally, the adhesive may fill cracks in the contact area between the vessels. Alternatively or additionally, the adhesive may serve to attach the two vessels together, for example being applied to one or both vessels prior to their being contacted or being injected into the contact points. In an exemplary embodiment of the invention, the adhesive used is one or more of Coseal, a binary sealant formed of two polyethylene glucol polymers, Costatis or Dynastat, by Cohesion Technologies, inc. (Palo Alto Calif.), Floseal, a collagen based matrix by Fusion medical technologies, inc. (Fremont Calif.), Tisseel, a fibrin sealant by Baxter (Deerfield Ill.), Hermaseel, by Haemacure (Kirkland, Canada), Cryoseal FS system by Thermogenesis (Rancho Cordova Calif.) and BioGlue, by Cryolife Inc. (Atlanta Ga.).

In the embodiment shown in FIG. 2F, the adhesive is provided via one or more nozzles 226. In an exemplary embodiment of the invention, nozzles 226 are used to provide a certain even or uneven distribution. Alternatively or additionally, if complex adhesive compounds are used, they may be mixed on site, for example, with different nozzles providing different components. Alternatively or additionally, the adhesives may be mixed before they exit the nozzle, for example in the nozzle or before the nozzle. Alternatively or additionally, one or more of the nozzles is used to provide an aggregate material, for example, fibers, to strengthen the anastomosis connection. In the embodiment shown, a split nozzle is used, which includes a slot 227 for receiving vessel 204.

Optionally, a frame, for example, a thread or a ring 242 is provided at the anastomosis location, for example, to assist in strengthening the adhesive or to assist in sealing the anastomosis. It should be noted that this frame on its own cannot form a patent anastomosis, and in some embodiments, is not attachable to blood vessels, except using adhesive.

FIG. 6 is a schematic illustration of an adhesive delivery system 600, in accordance with an exemplary embodiment of the invention. System 600 comprises a tube body having a splittable aperture 604, defined at its distal end, for passing vessel 204. A plurality of pullers 232 for pulling vessel 200 are shown extending form the distal end as well. In the embodiment shown, a pistol-grip 606, with a lever 608 for retracting pullers 232, is shown. In an exemplary embodiment of the invention, a knob 610 is provided for injecting adhesive, a scale for showing the amount of such injection 612 is optionally provided.

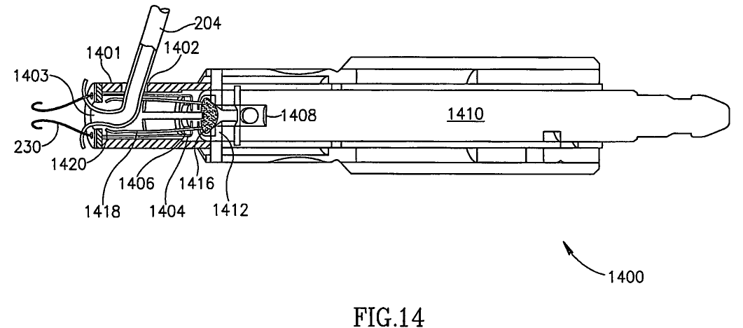

In an exemplary embodiment of the invention, the amount of adhesive applied is measured beforehand, for example, provided in a capsule of fixed and known size, for example as a collapsible capsule for example, underlying or in place of scale 612. Alternatively or additionally, the tip of system 600 is replaceable, for example as described in PCT application PCT/IL02/00790 and PCT publication WO 02/30172, the disclosures of which are incorporated herein by reference. In an exemplary embodiment of the invention, this tip includes an adhesive compartment. A replaceable tip capsule is described below in FIG. 14.

Alternatively, the lever is used for adhesive application and the knob for puller retraction. In an alternative embodiment of the invention, a hydraulic system is used for controlling the pullers and the adhesive application. A hydraulic system which is suitable (with appropriate modifications) is described in a US provisional application filed on Nov. 12, 2003, by inventor Amir Loshakove, et al., having the title “PRESSURE POWERED ANASTOMOTIC SYSTEM” and having attorney docket number [088/03773], the disclosure of which is incorporated by reference. In such a hydraulic system, a piston and cylinder mechanism is used in which fluid entering a chamber defined by the piston and the cylinder body causes motion of one or both of the cylinder body and piston, thereby acting like a retraction force on a capsule.

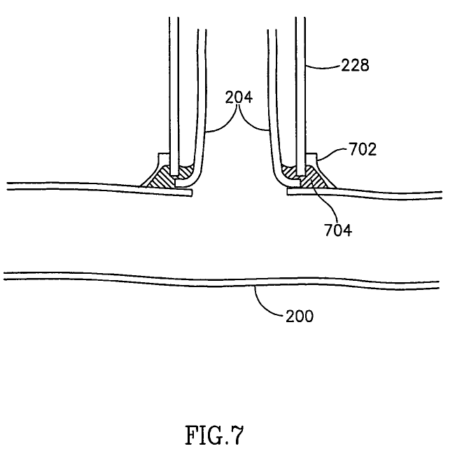

FIG. 7 shows an adhesive delivery system including an optional rim 702 which prevents spillage of adhesive out of the anastomotic area and/or shapes the geometrical distribution of the adhesive to a shape 704. Various rim shapes may be provided. The pullers are not shown, for clarity and may be contained, for example, in suitable slits and/or apertures.

Alternatively or additionally, to a rim, a separate form may be used. This form may be, for example, selected or distorted to match the particular anastomotic situation. Optionally, there is no openings between the form (or rim), the delivery system and the blood vessels, so that the anastomotic area can fill with adhesive and possible be pressured above atmospheric pressure by the nozzles.

In an exemplary embodiment of the invention, the inside of the delivery system is coated with or formed of a material which does not bond to the adhesive. However, in a one time use system this is not essential.

In an exemplary embodiment of the invention, the adhesive is provided in one or more sequential portions. Optionally, the delivery system is manipulated between sequential portions. In one embodiment of the invention, tube 228 is retracted after a first portion of adhesive is applied. In another embodiment, pullers 230 are retracted after a first portion of adhesive is applied, to pull vessel 204 into an area in which there is adhesive and/or ensure that there is a layer of adhesive between the vessels. Optionally, any adhesive that enters the blood vessels is washed away and diluted by the blood flow.

Alternatively to providing the adhesive as a fluid, it may be provided as a foam, for example using a foaming chamber (not shown) to convert fluid adhesive into a foam, for example using a gas source. Various gas sources are typically provided in operating rooms, including nitrogen, oxygen and carbon dioxide. Alternatively a dedicated canister may be provided. Alternatively, the adhesive is provided as a spray, for example, using an atomizing nozzle.

Optionally, the adhesive includes a clot-inducing material, or a healing-related material, or one may be provided as a primer.

While the adhesive is applied by a delivery system in exemplary embodiments of the invention, alternatively, the adhesive is applied by hand, for example being pre-applied to one or both of the vessels and/or applied using a syringe-like element.

Pre-Setting (118)

Prior to the adhesive setting (118), one or more acts are optionally carried out. In one example, excess adhesive is removed, for example by washing or by picking.

Setting (119)

During the setting time, heat, pressure and/or vibration are optionally applied to the adhesive, for example using delivery system 220 or a separate device, to assist in proper and/or rapid setting of the adhesive.

In an exemplary embodiment of the invention, heat is applied by providing an adhesive with small bubbles and applying ultrasonic radiation to the adhesive, to cause cavitation. Alternatively or additionally, one or more conducting wires are provided in the adhesive and which serve to receive RF radiation and heat by eddy currents.

In an exemplary embodiment of the invention, hot or unheated gas is blown through nozzles 226 or through a different set of nozzles. Cold gas is optionally used to reduce local blood flow. Alternatively or additionally, a separate device is used to blow the gas. In an exemplary embodiment of the invention, the gas is heated using an electrical heating element. The gas source may be, for example, a standard gas source as found in hospital rooms. Alternatively, a separate canister may be provided. Such a gas source may also be used for drying, for example as described above.

Optionally, gas blowing is used to blow away blood from leaks, thus possibly assisting in carrying out the procedure without blocking blood flow in vessel 200.

Blood flow (if blocked) may be enabled, for example, once the adhesive has sufficiently hardened (e.g., to bond and/or seal) and/or if the attachment (without adhesive) is strong enough. In some cases, blood flow may prevent adhesive from hardening inside the blood vessels.

Post-Setting (120)

While adhesive often asymptotically sets over a long period, a major portion of the setting is often completed within a short time, for example, 1 minute. After this time, the anastomosis may be considered to be “completed” for all or most intents.

Various methods may be provided for determining that the adhesive is set. In an exemplary embodiment of the invention, the adhesive hardens, which may be felt by manual manipulation. In another embodiment of the invention, a set time is allowed to pass before the procedure is considered to be completed. Optionally, the delivery system includes an electronic or mechanical timer, with an audio and/or visual alert to indicate the set time is over (e.g., indicated as an element 614 on FIG. 6). In another embodiment of the invention, the adhesive includes a compound or element that changes color or other optical properties such as diffusion or transparency, when the adhesive sets. Alternatively or additionally, a timer (e.g., electronic, mechanical or chemical) is inserted into the anastomosis area. Alternatively or additionally, a marker is inserted into the adhesive and is released by the setting of the adhesive. Optionally, one or more of pullers 230 or 232 serves as a marker, being coated for example with Teflon, to prevent adhesion.

Optionally, the delivery system is maintained at the anastomosis location until the adhesive is set. In an exemplary embodiment of the invention, a sensing method is used to determine the setting status, for example, by sensing electrical conduction or capacitance properties and/or using a pressure sensor (sensors not shown, but are provided for example near the nozzles).

Optionally, the anastomosis area is shaped, for example by removing excess adhesive and/or smoothing any sharp edges inadvertently formed during or after setting.

Scaffold Removal (122)

In some embodiments of the invention, the scaffold dissolves or is absorbed after a while. Alternatively, the scaffold may be kept in the body. Alternatively, the scaffold is removed, to reduce the existence of foreign materials in the body. In an exemplary embodiment of the invention, the scaffold parts are simply pulled away from the vessels. Optionally, the scaffold is heated or vibrated, to assist in removal.

FIG. 2G shows a completed anastomosis, in accordance with an exemplary embodiment of the invention, including an adhesive section 250, and in which the pullers were retracted so that the two vessels touch tip to tip.

Optionally, if the scaffold hole leaks, more adhesive is applied. Alternatively or additionally, compression is applied until the leaking blood clots.

In an exemplary embodiment of the invention, the delivery system is a split system, for example as described in PCT publication WO 00/56226, the disclosure of which is incorporated herein by reference, in which is split for removal from vessel 204.

Post-Procedure (124)

After the anastomosis procedure is completed, a check for leaks is optionally performed (any leaks are optionally corrected by applying a dab of adhesive) and the entry port is closed. After a period of time, the adhesive optionally dissolves or is absorbed, leaving no foreign materials in the body, in accordance with some embodiments of the invention.

Exemplary Adhesive Materials

A wide range of adhesive materials may be used for the above described embodiments. In some cases, the delivery system will be modified, for example, to account for differing viscosity, drying times, primers and/or drying enhancers suitable for each adhesive. In an exemplary embodiment of the invention, the adhesive is selected to dissolve or otherwise dissipate after between 1 day and 2 weeks, for example, between 3-7 days. In some types of adhesives, the tissue grows into or around the adhesive, mooting the need for an adhesive. For example, matrix type adhesives tend to encourage tissue grows into the matrix. Alternatively, the two blood vessels are contacted and at the contact point tissue adhesion or ingrowth occurs. Optionally, no adhesive is provided at those points, for example, by first contacting the vessels with a minimum force to ensure good contact and then applying adhesive, which does not flow between the contact surfaces. In other embodiments, the adhesive is of a type that flows between contact surfaces or the contact pressure is low enough to allow such flow in a regular adhesive.

Approximation Geometries

The lips of the two vessels may be arranged in various ways, each of which may have utility for certain situations and/or conditions, for example, one or more of the following considerations may be relevant:

(a) minimizing non-intima surfaces may provide best blood interface;

(b) minimizing stretching of vessels may prevent tears and/or distortion of an anastomosis area during and after procedure;

(c) maximizing contact area between vessels may reduce leaks or enhance strength; and

(d) laying out of vessels in a manner which minimizes contact (or makes it easier to minimize) between the anastomosis area and nearby tissue during the setting period.

In contrast to general anastomotic connectors, in an exemplary embodiment of the invention, some freedom of approximation configuration is provided by the adhesive optionally sealing in the entire anastomosis area, possibly relieving strain, preventing leaks and/or strengthening the connection between the blood vessels.

Optionally, a single delivery device is used to provide multiple arrangements, for example, based on how far and/or relative retraction of pullers and/or based on how scaffolds are mounted on the blood vessels.

In the example of FIG. 2, the relative positions of the two blood vessels can optionally be controlled by selectively pulling back more on one or the other of pullers 232 and 230. Optionally, the handle of the delivery system includes one or more settings for relative positions of the two sets of pullers, when retracted.

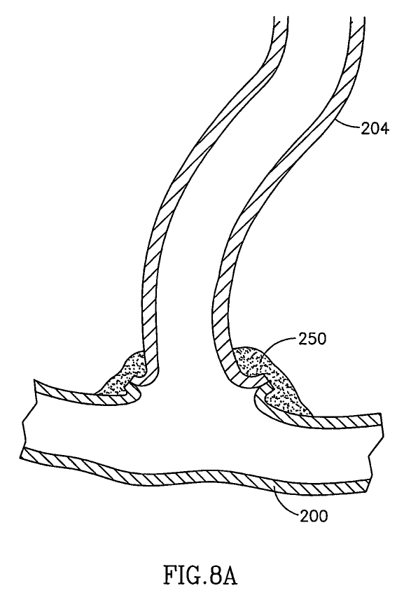

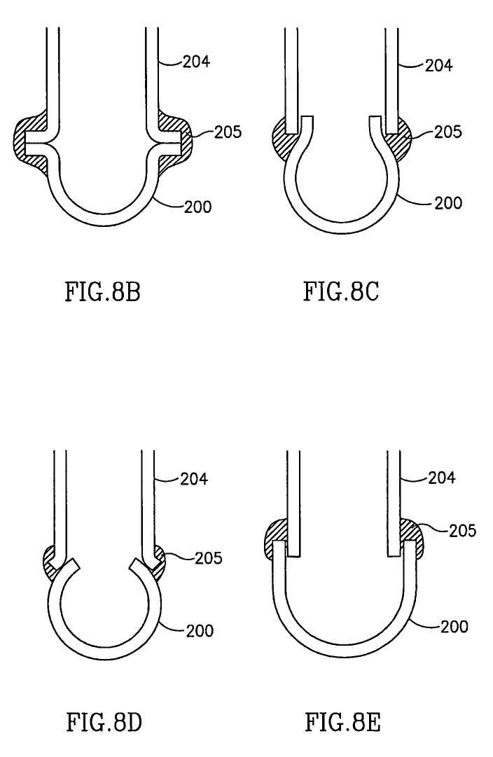

FIGS. 8A-8E show various attachment configurations of blood vessels, in accordance with exemplary embodiments of the invention. FIG. 8A shows an intima-to-intima connection. FIG. 8B shows another intima-to intima connection, in which vessel 200 is everted more and vessel 204 less, than in FIG. 8A. FIG. 8C shows a configuration where the lips of vessel 200 are inserted inside vessel 204. FIG. 8D shows an embodiment, where only vessel 204 is everted and only a small amount (alternatively, no eversion is provided). FIG. 8E shows an embodiment where vessel 204 is inserted into vessel 200.

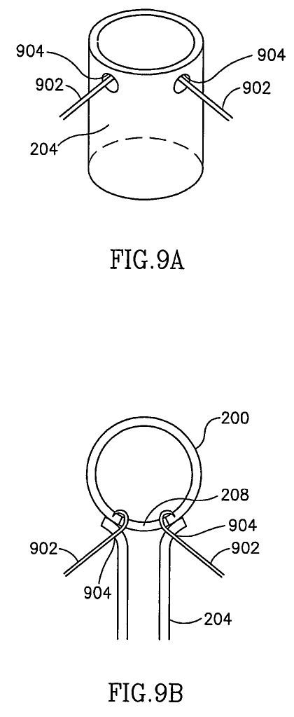

FIGS. 9A and 9B show an exemplary method of achieving the configuration of FIG. 8C, in accordance with an exemplary embodiment of the invention. In this embodiment, two or more pullers 902 are provided through one or more matching apertures 904 that are formed in vessel 204. FIG. 9B shows how vessel 200 is engaged on its inside by pullers 902. When pulled, the configuration of FIG. 8C results. The holes left by pullers 902 in vessel 204 may be self-sealing. Alternatively or additionally, a dab of adhesive is applied. Alternatively or additionally, an adhesive patch is applied. A patch of vascular tissue or other material is provided over the anastomosis area, in some embodiments of the invention.

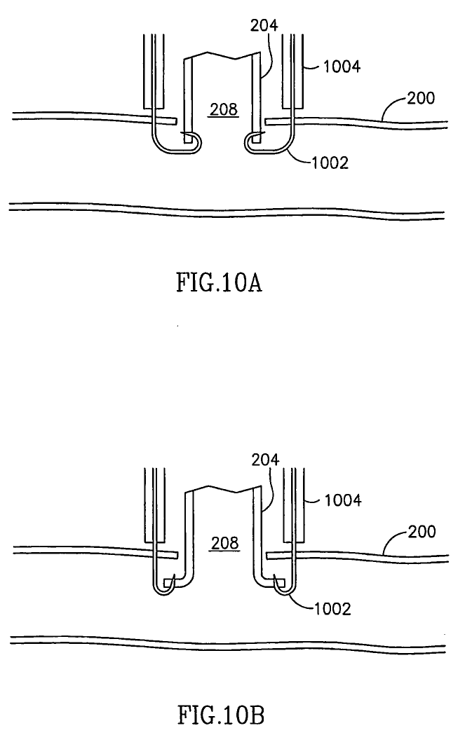

FIGS. 10A and 10B show an exemplary method of achieving the configuration of FIG. 8E, in accordance with an exemplary embodiment of the invention. A plurality of pullers 1002 penetrate through a wall of vessel 200 and engage vessel 204, after it is inserted into incision 208, from the inside. In an exemplary embodiment of the invention, pullers 1002 are pre-stressed to be bent and are inserted using a plurality of over-tubes 1004. During insertion puller 1002 is completely enclosed by the over tube except for its tip. This tip penetrates vessel 200 and then, as it is extended from overtube 1004, curls and engages vessel 204. Optionally, a contra (not shown), for example a slotted tube provided over vessel 204 is used to maintain vessel 204 open and assist in penetration of pullers 1002 into vessel 204. In FIG. 10B, pullers 1002 are retracted, so that they straighten and flare vessel 204 as shown. If retracted a small amount, vessel 204 will be partially everted/flared and vessel 200 not everted. If pulled back more, a configuration similar to that of FIG. 8E will result.

Thus, FIGS. 9 and 10 show that a single set of pullers can be sufficient for approximation and attachment. It should be noted that by utilizing a flared element such as shown in FIGS. 4E or 5C, the pullers can be retracted in various directions and amounts. While pullers are mentioned, similar methods can be applied for scaffolds of various types.

In some embodiments of the invention, the pullers are pre-bent and/or pre-strained (e.g., for super elastic elements) by hand or machine to match a desired angle of the anastomosis.

Nozzle Designs

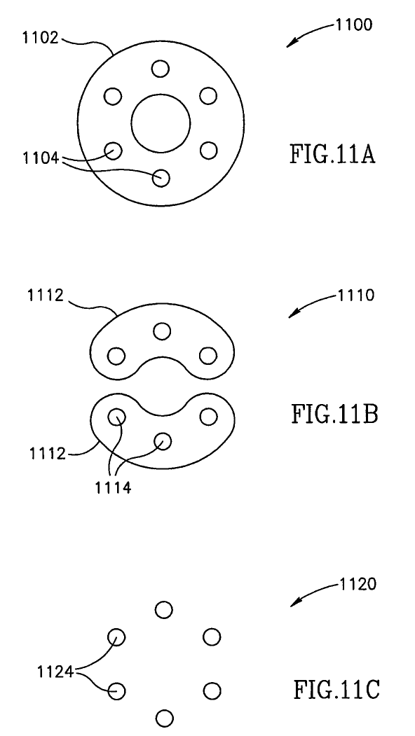

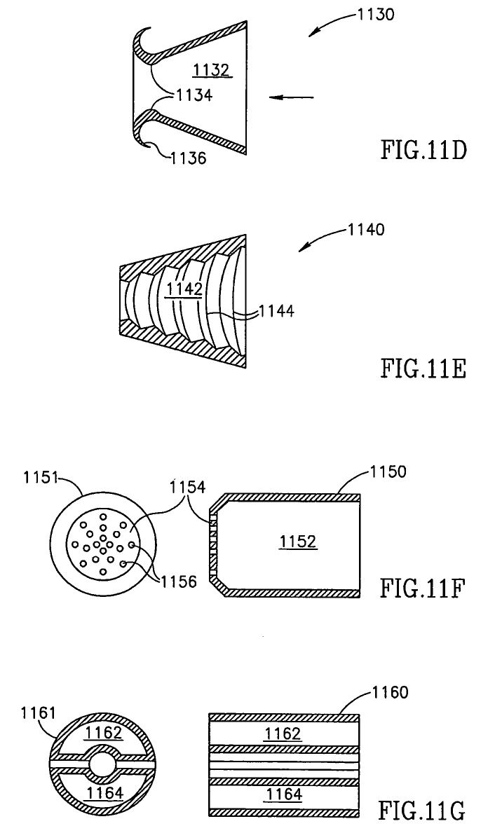

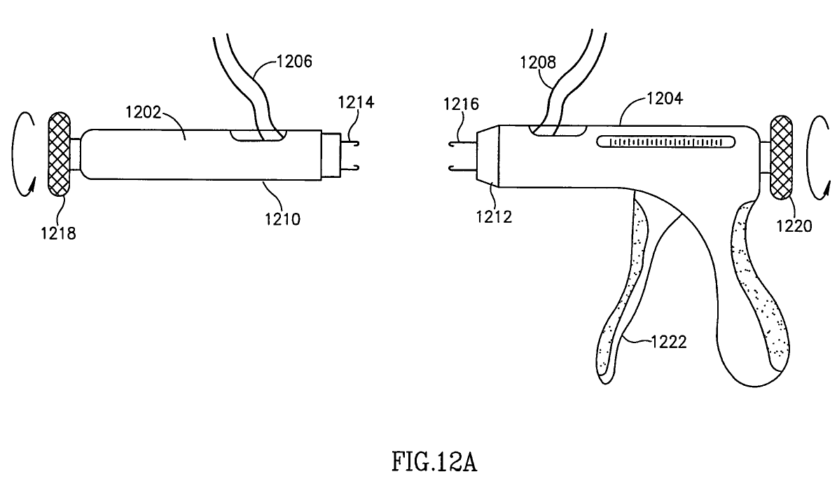

FIGS. 11A-11G show nozzle designs, in accordance with exemplary embodiments of the invention, of which, FIGS. 11A-11C show nozzle set designs.

FIG. 11A is a front view of a nozzle set 1100, in which a ring shaped canister 1102 (optionally elongated like a cylinder) defines a plurality of nozzles 1104 for providing adhesive or other materials. Vessel 204 (not shown) optionally passes through the hole of the ring. A slotted ring may be provided (not shown) with the vessel fitting through the slot.

FIG. 11B shows an alternative nozzle set 1110, in which two separate arcuate shapes 1112 each define a plurality of nozzles 1114. Optionally, each shape 1112 is used for delivering a different material. Alternatively or additionally, each shape may be positioned at a different axial position and/or may be controlled separately, for example, to have different adhesive extrusion rates.

FIG. 11C shows a nozzle set 1120, in which a plurality of nozzles 1124 are each fed by a separate tube (not shown).

A mixture of different nozzle arrangements may be provided as well. Where a single nozzle is used for delivering a plurality of fluid types, the nozzle may include a switch (not shown) for switching between different sources. Different nozzles may have different shapes, types, locations (axial), angles (to the device axis), sources, material and/or pressure feed. For example, a set of nozzles may include alternating adhesive and setting material nozzles.

FIGS. 11D-11G show individual nozzle designs in accordance with exemplary embodiments of the invention.

FIG. 11D is a cross-sectional view of a nozzle 1130, having a mixing area 1132 and a pre-exit narrowing 1134. A flare 1136 is provided, which may, for example as shown, curl back. This may assist in forming the adhesive into drops.

FIG. 11E shows a mixing nozzle 1140, in cross-sectional view. A mixing area 1142 includes a plurality of threads or baffles 1144, which cause turbulence and mixing of the material in the nozzle.

FIG. 11F shows a watering-can like nozzle 1150 in side cross-section and front views. The nozzle comprises a mixing area 1152 and a front plate 1154 defining a plurality of apertures 1156 (shown in frontal view 1151).

FIG. 11G shows a paired nozzle 1160, in which two arc-profile lumens 1162 and 1164 are provided. Nozzle 1160 may be solid at its center or hollow as shown. In an exemplary embodiment nozzle 1160 is large (as shown for example in a front view 1161) enough to include a vessel at its center, in which case one or more slots may be provided for receiving the vessel.

Exemplary End-to-End Embodiment

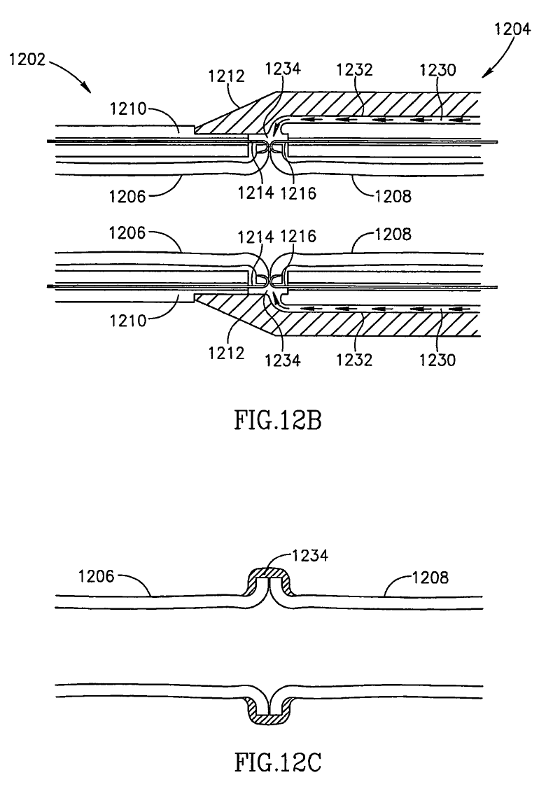

FIGS. 12A-12C illustrate an end-to-end anastomosis, in accordance with an exemplary embodiment of the invention. In this embodiment, two end vessels 1206 and 1208 are attached end to end. Each vessel is mounted on its own delivery system, 1202 and 1204 respectively, which are optionally adapted to interlock, for example, by an inner tube 1210 of delivery system 1202 fitting inside an outer tube 1212 of delivery system 1204. Other interlocking mechanisms may be used as well. A fixing means, for example threading, friction or a clasp, are optionally provided.

In the embodiment shown, the vessels enter through the side of the delivery systems and pullers 1214 and 1216 are provided for everting the vessels, as shown in FIG. 12B. Knobs 1218 and 1220 are optionally provided for retracting the pullers. Adhesive is optionally provided by only one of the systems, for example, system 1204, using a lever 1222, for example.

FIG. 12B is a side cross-sectional view, showing systems 1202 and 1204 interlocked and showing the vessels retracted and everted by the pullers, so that they contact intima-to-intima Also shown is provision of adhesive 1230 via a plurality of channels 1232 to an anastomosis region 1234 which is defined by the interlocking systems.

FIG. 12C shows the end result of the anastomosis, in side cross-sectional view.

A similar system can be used for side to side or side to end connections. A portion that holds a side vessel will generally not be rotationally symmetric, however.

Internal Support Embodiment

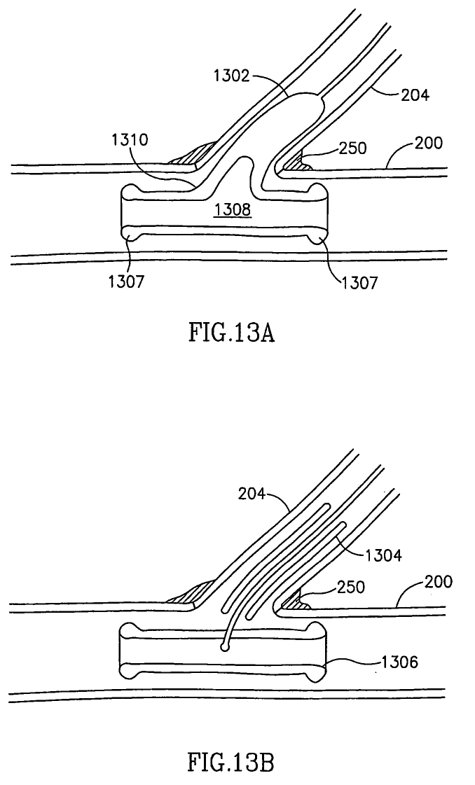

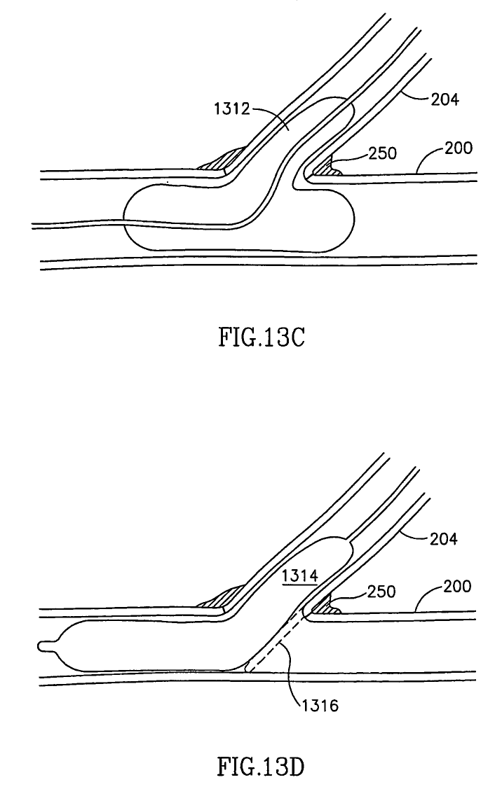

FIGS. 13A-13D are side cross-sectional views showing embodiments in which a support is provided inside the blood vessels, alternatively or additionally to using scaffolding and/or pullers during the application of adhesive. The shown structures are optionally provided through a hole in one or both vessels, which may then be closed, for example with a tissue patch, with a closure device or with a suture. In an exemplary embodiment of the invention, existing or modified bypass shunts are used. Such shunts are sold for example under the trade name Flo-Through, by Bio-Vascular Inc., which also sells occluders, under the name Flo-Rester.

In FIG. 13A, a light collapsible structure 1302 is provided, which, when retracted, can be pulled out through end vessel 204. Since this structure only provides some support during the anastomosis procedure, it is generally not required to be strong. As shown, a lumen 1308, which may be used to shunt blood is provided. Alternatively, no lumen (and thus no blood flow) is provided. In an exemplary embodiment of the invention, an area 1310 of the structure which is near the anastomotic location, is substantially in contact with the location (not as shown, for clarity), to provide support for the anastomosis area. In standard shunts and occluders, such contact may be undesirable, as it increases device size and may interfere with manipulation of the vessel (or sutures). It should also be noted that in some embodiments of the invention blood flow near the anastomosis location is allowed before, during and/or after the adhesive is applied, so a complete seal of the device (e.g., of one or both lips 1307 thereof) to the vessel walls is not required in these embodiments and unlike a shunt or occluder.

In FIG. 13B, two separate elements are provided, a light structure 1306 (e.g., shunt or occluder) which may be retracted through a hole in side vessel 200 and a tube 1304 which is retracted through vessel 204 (and may support vessel 204 during anastomosis). Optionally, structure 1306 is retracted and collapsed into tube 1304, for removal.

In FIG. 13C, a single, T-shaped balloon 1310 provides support.

In FIG. 13D, a regular balloon 1314 is used for support, with vessel 200 acting as a continuation of vessel 204 after the anastomosis. Possibly, one branch of vessel 200 is sealed by the anastomosis, but this is not essential, for example if a wall 1316 of vessel 204 continues into vessel 200.

In some embodiments of the invention, the structures of FIGS. 13A-13D are fixedly coupled to the adhesive delivery system, for example by providing suitable jaws (not shown) on the delivery system.

Automation and Feedback

In an exemplary embodiment of the invention, the adhesive delivery system is provided with various automatic features (some of which are described above), for example, one or more of:

(a) providing feedback on setting of adhesive, on heating of adhesive and/or on drying of anastomosis area;

(b) safety features, such as some controls being active only after a previous control was used, for example, adhesive flow being enabled only after puller retraction;

(c) electrical power instead of manual power, for example for adhesive injection, for example, to reduce jumping and/or provide finer control over adhesive flow; and

(d) passage of time indicator(s).

The linkage between controls may be, for example, electrical. Alternatively or additionally, the linkage is mechanical, for example, retraction of pullers freeing a pin that allows an adhesive provision lever to be moved.

Capsule Embodiment

PCT/IL02/00790 and WO 02/30172, the disclosures of which are incorporated herein by reference, describes a capsule based delivery system in which a removable capsule is attached on a delivery system and the capsule includes both a connector and a mechanism for deploying that connector. FIG. 14 shows a variation of such a capsule 1400, in cross-sectional view, showing the integration of an adhesive delivery system into such a capsule. The connector, as shown may be deployed by the capsule, in one embodiment of the invention. In an alternative embodiment of the invention, which may be preferred, for example since fewer foreign materials are left in the body, the connector may be used as mere scaffolding to be retracted with the system after the adhesive at least partly sets.

Capsule 1400 comprises a forward end 1401 having an aperture 1402 through which a vessel 204 enters end 1401, to exit from a front aperture 1403. A plurality of pullers 230 are shown being an extension of a base portion 1404 (which in an alternative design may be backward spikes of an anastomotic connector). When a piston 1410 (or other mechanism) of capsule 1400 is retracted, a lever 1408 is retracted, which lever is coupled to a base 1406 on which base portion 1404 rests. This causes retraction of pullers 232. At the same time, a bladder of adhesive 1416 is compressed between base 1406 and a narrowing 1412 (which may be flared rather than stepped as shown) of capsule 1400. Adhesive is thereby forced to flow through tubes 1418 to delivery nozzles 1420, adjacent the anastomosis area.

As can be appreciated such capsule may be used for various types of procedural approaches, for example, open surgery, catheter based surgery, endoscopic surgery or keyhole surgery.

It should be appreciated that also a non-capsule delivery system may be modified as described above to provide adhesive to an anastomosis area, for example in synchrony with retraction, as described, or at least partially independent of it, using a second lever.

Connector and Anastomosis Tools

As noted above, embodiments of the present invention may be used with various types of anastomotic connectors, anastomosis assisting tools and delivery systems. In particular, the following documents, describe connectors, delivery systems and/or other tools and methods which are useful in conjunction with embodiments of the prevent invention:

PCT/IL02/00790, filed on Sep. 25, 2002, now published as WO 03/026475;

PCT/IL02/00215, filed on Mar. 18, 2002, now published as WO 02/074188;

PCT/IL01/01019, filed on Nov. 4, 2001, now published as WO 02/47532;

PCT/IL01/00903, filed on Sep. 25, 2001 now published as WO 02/30172;

PCT/IL01/00600, filed on Jun. 28, 2001, now published as WO 02/47561;

PCT/IL01/00267, filed on Mar. 20, 2001, now published as WO 01/70091;

PCT/IL01/00266, filed on Mar. 20, 2001, now published as WO 01/70090;

PCT/IL01/00074, filed on Jan. 25, 2001, now published as WO 01/70119;

PCT/IL01/00069, filed on Jan. 24, 2001, now published as WO 01/70118;

PCT/IL00/00611, filed on Sep. 28, 2000, now published as WO 01/41624;

PCT/IL00/00609, filed on Sep. 28, 2000, now published as WO 01/41623,

PCT/IB00/00310, filed on Mar. 20, 2000, now published as WO 00/56228;

PCT/IB00/00302, filed on Mar. 20, 2000, now published as WO 00/56227;

PCT/IL99/00674, filed on Dec. 9, 1999, now published as WO 00/56223;

PCT/IL99/00670, filed on Dec. 8, 1999, now published as WO 00/56226;

PCT/IL99/00285, filed on May 30, 1999, now published as WO 99/62408; and

PCT/IL99/00284, filed on May 30, 1999, now published as WO 99/62415. The disclosure of all of these applications, which designate the US and were filed in English, are incorporated herein by reference.

In addition, the following PCT unpublished applications, all filed on Sep. 25, 2003, in English and designating the US, the disclosures of which are incorporated herein by reference describe tools and connectors which may be useful: PCT/IL03/00774, “Anastomotic Connectors”, PCT/IL03/00773, “Snare”, PCT/IL03/00770, “Sliding Surgical Clip”, PCT/IL03/00771, “Blood Vessel Cutter”, and PCT/IL03/00769, “Anastomotic Leg Arrangement”.

The following US provisional applications, the disclosures of which are incorporated herein by reference, also describe tools which may be of use, U.S. Ser. No. 60/492,998 filed on Aug. 7, 2003 and U.S. Ser. No. 60/505,946, filed on Sep. 25, 2003 (in which an adhesive feature may be added in accordance with an exemplary embodiment of the invention).

Some of these applications describe anastomosis delivery systems, hole making apparatus and/or other connectors useful in cooperation with the present invention.

While the above delivery system has been described in general for any type of blood vessel, it should be appreciated that particular modifications may be desired for certain vessel types. For example, the aorta is thicker, while a coronary vessel is thinner, thus suggesting different vessel holder sizes and/or puller geometries. For example, an aorta may be 3 mm thick, while a coronary vessel may be less than 1 mm thick.

It should be noted that the term “connector” should be construed broadly to include various types of connectors, including one part, two part and multiple part connectors, some of which when deployed, result in a plurality of individual clip-like sections.

The term “eversion”, where used means not only complete eversion of 180 degrees, but also partial eversion or flaring, for example of 90 degrees. Also, in some embodiments, mounting without eversion is provided.

Measurements are provided to serve only as exemplary measurements for particular cases. The exact measurements stated in the text may vary depending on the application, the type of vessel (e.g., artery, vein, xenograft, synthetic graft), size of connector, shape of hole (e.g., incision, round) and/or sizes of vessels involved (e.g., 1 mm, 2 mm, 3 mm, 5 mm, aorta sized).

While the term “tube” and other geometrical shapes have been described and used for generality, it should be appreciated that this tube need not have a full body nor have a circular cross-section, in some embodiments.

It will be appreciated that the above described methods of adhesive anastomosis may be varied in many ways, including, changing the order of steps and the types of tools used. In addition, a multiplicity of various features, both of method and of devices have been described. In some embodiments mainly methods are described, however, also apparatus adapted for performing the methods are considered to be within the scope of the invention. It should be appreciated that different features may be combined in different ways. In particular, not all the features shown above in a particular embodiment are necessary in every similar embodiment of the invention. Further, combinations of the above features, also for different embodiments, are also considered to be within the scope of some embodiments of the invention. Also within the scope of the invention are surgical kits which include sets of medical devices suitable for performing, for example, a single or a small number of anastomotic procedures. In some embodiments, one or more of the devices, generally sterilize, described above, are packaged and/or sold with an instruction leaflet, describing the device dimensions and/or situations for which the device should be applied. Section headings where are provided are intended for aiding navigation and should not be construed to limiting the description to the headings. When used in the following claims, the terms “comprises”, “includes”, “have ” and their conjugates mean “including but not limited to”.

It will be appreciated by a person skilled in the art that the present invention is not limited by what has thus far been described. Rather, the scope of the present invention is limited only by the following claims.

1. A method of performing an anastomosis, comprising:
juxtaposing two blood vessels to be anastomosed using an juxtaposition device, to a desired configuration in which at least one vessel is an end vessel;
applying an adhesive to said vessels while they are in said configuration, said adhesive being sufficient to ensure both sealing and bonding of said two vessels to each other; and
removing said juxtaposition device after said adhesive sufficiently sets.

2. (canceled)

3. A method according to claim 1, wherein juxtaposing comprises inserting at least a portion of said juxtaposing device into at least one of said vessels.

4. A method according to claim 1, wherein juxtaposing comprises inserting at least a portion of said juxtaposing device into a wall of at least one of said vessels.

5-17. (canceled)

18. A method according to claim 1, wherein applying comprises applying using a plurality of nozzles arranged in a ring.

19. A method according to claim 1, wherein applying comprises applying into a mold adjacent said anastomosis area, which mold shapes the adhesive about said area

20. (canceled)

21. A method according to claim 1, wherein applying comprises applying a pre-measured amount of adhesive.

22-24. (canceled)

25. A method according to claim 1, wherein said removing comprises removing before said adhesive sets completely.

26. A method according to claim 1, wherein said removing comprises removing as soon as said adhesive starts to set.

27-28. (canceled)

29. A method according to claim 1, wherein no foreign materials other than said adhesive remain in said anastomosis.

30. A method according to claim 1, comprising providing at least one strengthening element in said anastomosis and leaving said at least one strengthening element permanently in said anastomosis.

31. A method of performing an anastomosis, comprising:
attaching at least a first scaffold element to a first blood vessel;
attaching at least said first scaffold element or a second scaffold element to a second blood vessel;
positioning said blood vessels using said at least a first scaffold element, to a desired configuration;
applying an adhesive to said vessels while they are in said configuration, said adhesive being sufficient to ensure both sealing and bonding of said two vessels to each other; and
removing said at least first scaffolding element.

32. A method according to claim 31, wherein said first blood vessel is a side vessel.

33. A method according to claim 31, wherein said first blood vessel is an end vessel.

34. A method according to claim 31, comprising mechanically attaching said at least first scaffolding element to said first blood vessel.

35. A method according to claim 34, wherein mechanically attaching comprises piercing.

36. A method according to claim 34, wherein mechanically attaching comprises hooking.

37-40. (canceled)

41. A method according to claim 31, wherein said at least one scaffolding element comprises a plurality of wires adapted to engage a blood vessel.

42. (canceled)

43. A method according to claim 31, wherein said at least one scaffolding element comprises a balloon.

44-50. (canceled)

51. A method according to claim 31, wherein removing comprises removing at least one of said at least one scaffolding elements before said adhesive sets.

52-76. (canceled)

77. An adhesive anastomotic system, comprising:
a first blood vessel holder;
a second blood vessel holder adapted to interlock with said first blood vessel holder, such that blood vessels held by said two vessel holders contact; and
an adhesive port configured to deliver an adhesive to said contact.

78-81. (canceled)
